(19) United States
(12) Patent Application Publication
(10) Pub. No.: US 2024/0350709 A1
(43) Pub. Date: Oct. 24, 2024

(54) MULTISTAGE SUSPENSION PRINTING METHOD FOR CONSTRUCTING COMPLEX HETEROGENEOUS TISSUE/ORGAN

(71) Applicant: TSINGHUA UNIVERSITY, Beijing (CN)

(72) Inventors: Zhuo XIONG, Beijing (CN); Yongcong FANG, Beijing (CN); Ting ZHANG, Beijing (CN); Yihan GUO, Beijing (CN); Yujiang GUO, Beijing (CN)

(21) Appl. No.: 18/560,576

(22) PCT Filed: Aug. 20, 2021
(86) PCT No.: PCT/CN2021/113653

§ 371 Date: Apr. 24, 2024

(30) Foreign Application Priority Data
May 14, 2021 (CN) 202110526251.3

(51) Int. Cl. A61L 27/38; A61F 2/06; A61L 27/22; A61L 27/50; A61L 27/52; B33Y 10/00; B33Y 40/20; B33Y 70/00; B33Y 80/00

(52) U.S. Cl. CPC A61L 27/38; A61F 2/06; A61L 27/222; A61L 27/507; A61L 27/52; B33Y 10/00; B33Y 40/20; B33Y 70/00; B33Y 80/00; A61F 2240/002; A61L 2400/06

(57) ABSTRACT

A multistage embedded printing method for constructing a complex heterogeneous tissue/organ includes preparing a bioink formed from a cell-laden gel microsphere which is cross-linked, or is obtained by mixing a cell-laden gel microsphere which is cross-linked with a gel material which is not cross-linked. The method includes printing the bioink in a suspension medium to construct a specific construction of tissue/organ. The method includes further second stage or multistage substructure printing within the construction of the tissue/organ structure. The method includes, after printing, dissolving out the suspension medium after a whole cross-linking to obtain it. The multistage suspension 3D printing method uses a gel microsphere ink with both shear thinning and self-healing property, which can be printed in a suspension medium and then used as a suspension medium for a next stage structure printing, which is suitable for constructing a tissue/organ model with vascularized channel and a heterogeneous cellular structure.

### RELATED APPLICATIONS

The present application is a U.S. National Phase of International Application Number PCT/CN2021/113653 filed Aug. 20, 2021, which claims priority to Chinese Application Number 202110526251.3 filed May 14, 2021.

### TECHNICAL FIELD

The present invention relates to a multistage suspension printing method for constructing complex heterogeneous tissue/organ, and belongs to technical field of tissue engineering and biological manufacturing.

### BACKGROUND

Tissue engineering and regenerative medicine, as an emerging interdisciplinary discipline, aims to construct artificial tissue and organ with bionic structure and function in vitro, which has broad application prospects in the fields of regeneration and repair of tissue and organ, development and screening of drug, and construction of disease modeling, etc. At present, tissue-engineered products, such as bladders, skin, cartilage, and blood vessels, are currently applicable for medical use; however, construction of a complex heterogeneous tissue/organ in vitro, such as heart and liver, is still in its infancy.

A top-down strategy is mainly adopted in traditional tissue-engineered methods, represented by a cell-scaffold composite technology, in which a construction is directly constructed by a composition of cells and porous scaffolds, and then a functional maturation of the construction is induced by cell assembling and extracellular matrix remodeling; however, this strategy faces challenges, such as uneven cell distribution, low seeding efficiency, and heterogeneous cell seeding difficulty, which makes it difficult to construct the complex heterogeneous tissue and organ. In recent years, with a rapid development of biofabrication technology, a bottom-up strategy of constructing tissue and organ has been widely adopted in the field. The bottom-up strategy is represented by a 3D bioprinting, in which tissue and organ with complex structures are formed by depositing a cell-laden bioink in a layer-by-layer fashion according to a predefined path. Among them, the micro-extrusion printing method has become a main current 3D bioprinting technique due to its wide applicable range of biomaterial. However, due to poor mechanical property of a hydrogel, it is difficult to directly print non-self-supporting structure such as a hollow shell, an overhanging beam, and a convolution, so that their application to construction of a complex tissue and organ is limited.

One strategy is hydrogel enhancement, that is, synthetic polymer construction with high-strength is introduced to provide necessary support for printing a cell-laden bioink, as represented by the work of Kang's group in Korea (Kang, H. et al. A 3D bioprinting system to produce human-scale tissue constructs with structural integrity. Nature Biotechnology, 2016, 34, 312-319.) They developed a multi-nozzle printing device that combines cell-extrusion printing technique and Fused Deposition Molding technique to successfully print tissue such as a mandible, an ear cartilage, and a skull, etc. Although structural support for a hydrogel is provided and precise control of cell deposition is allowed according to this strategy of hydrogel enhancement, space available for tissue maturation is badly limited due to a relatively low printing accuracy of a polymer (≈200 μm), while a stiffer polymer material is not suitable for constructing a soft tissue, such as a heart.

Another strategy is suspension printing, that is, support for a printed structure is depended on a suspension medium to enable the shaping of highly complex structures (McCormack, A., Highley, C. B., Leslie, N. R. & Melchels, F. P. W. 3D bioprinting in suspension baths: keeping the promises of bioprinting afloat. Trends in Biotechnology, 2020, 38, 584-593). In addition, use of low-viscosity bioink of extracellular matrix material, such as collagen and fibrin, is allowed by suspension printing, such that a more suitable micro environment is provided for functional maturation of the tissue and organ. For example, a cardiac model containing vascular structure was printed by using bioink prepared with acellular extra matrix according to a strategy of suspension printing by Tal Dvir's group in Israel in 2019 (Noor, N., et al., 3D printing of personalized thick and perfusable cardiac patches and hearts. Advanced Science, 2019. 6(11): p. 190034.); although suspension printing with a variety of inks was achieved in this study, it is difficult to construct a structural feature (e.g., micro-vessels and nerves) with precision of one hundred micrometers or less, such that its application in the term of the bionic construction of complex tissue and organ is limited.

In summary, 3D bioprinting technology has tremendous advantages in term of construction of tissue/organ; however, a key challenge in the field of regenerative medicine is remained to construct a tissue/organ with sophisticated vascularized channel and heterogeneous cellular architecture in vitro, which greatly limits its application in the field of translational medicine.

### SUMMARY

The purpose of the present invention is to provide a new multistage suspension 3D printing method that utilizes the shear-thinning and self-healing property of cell-laden gel microsphere bioink, which can be printed and shaped in a suspension medium, and at the same time can be applied as a suspension medium for a next stage of printing, providing a novel method for constructing a complex heterogeneous tissue/organ, and which has an important medical application prospect in the translational and clinical field.

The multistage suspension 3D printing method for constructing a tissue/organ model with a sophisticated vascularized channel and/or a heterogeneous cellular architecture provided by the present invention comprises the following steps:
S1, preparing a bioink, the bioink is formed from a cell-laden gel microsphere which is cross-linked, or is obtained by mixing a cell-laden gel microsphere which is cross-linked with at least one gel material which is not cross-linked;

When both are included, the cell-laden gel microsphere acts as a dispersed phase and the gel material acts as a continuous phase;
S2, printing the bioink in a suspension medium to construct a construction of specific tissue/organ;
S3, processing a second stage or multistage printing within the construction of the tissue/organ obtained in step S2 to form substructure;
S4, at the end of printing, the suspension medium is dissolving after cross-linking the whole construction, so as to obtain a tissue/organ model with a sophisticated vascularized channel and/or a heterogeneous cellular architecture.

In above method step S1, the cell-laden gel microsphere is prepared according to the following method:

At least one of suspension droplet method culture, ultra-low adherence culture plate, magnetic suspension culture, dynamic rotary culture and microfluidic technology;

The cell is at least one of pluripotent stem cell, induced pluripotent stem cell, various tissue parenchymal cell, angiogenic cell, stromal cell and tumor cell.

The cell density in the cell-laden gel microsphere is 10⁶ cells/mL to 10⁸ cells/mL, specially is 1×10⁶ cells/mL to 1×10⁷ cells/mL, 1×10⁷ cells/mL, 2×10⁶ cells/mL or 5×10⁶ cells/mL;

The mass-volume concentration of the gel material as the continuous phase is 1 to 100 mg/mL, such as 20 to 50 mg/mL.

In above method step S1, both the gel material which is used for the cell-laden gel microsphere and the gel material which is used as the continuous phase are natural polymer hydrogel and/or synthetic polymer hydrogel;

The natural polymer hydrogel is at least one of sodium alginate, gelatin, collagen, Matrigel, chitosan, filipin, hyaluronic acid, fibrinogen, chondroitin sulfate, albumin, and their methacryloylated products (such as methacryloylated gelatin (GelMA), methacryloylated sodium alginate (AlgMA), etc.);

The synthetic polymer hydrogel is at least one of polyethylene glycol (PEG), polypropylene alcohol (PVA), polyethylene glycol diacrylate (PEGDA), polyethylene oxide (PEO), polyacrylamide (PAM), polyacrylic acid (PAA), polyphosphonitrile (PAMPS), poly-N-isopropylacrylamide-type hydrogels (PNIPAAm), and their methacryloylated products (such as concave-arm polyethylene glycol acrylate (4-arm-PEG-AC), methacryloylated polyvinyl alcohol (PVAMA), etc.).

The cell-laden gel microsphere has a size (diameter) of 50 μm to 1000 μm, such as 100 μm to 150 μm, 400 μm to 450 μm, or 450 μm to 500 μm, and may have a volume content of 40% to 100% in the bioink, and for example, when the volume content is 100%, the 3D printing bioink is formed from the cell-laden gel microsphere only.

In above method step S1, the mass-volume concentration of the gel material as the continuous phase may be 1 to 100 mg/mL, such as 4 to 25 mg/mL;

The gel material which acts as the continuous phase may lade cell;

The cell density in the gel material is 10⁶ cells/mL to 5×10⁷ cells/mL, such as 1×10⁷ cells/mL to 5×10⁷ cells/mL.

In above method step S2, the suspension medium is a hydrogel material with self-healing property, specifically is a supramolecular self-healing hydrogel and/or a microgel construction;

The supramolecular self-healing hydrogel is at least one of a cyclodextrin-based supramolecular hydrogel, a DNA supramolecular hydrogel, a polyurethane urea supramolecular hydrogel, a hyaluronic acid-dextran supramolecular hydrogel, a tanshinone II-A polypeptide supramolecular hydrogel, and a graphene composite supramolecular hydrogel;

The microgel construction has a size of 1 μm to 50 μm;

The microgel construction is at least one of Carbomer, gelatin and sodium alginate.

Specially, the Carbomer is an acrylic cross-linking resin obtained by cross-linking pentaerythritol with acrylic acid, and solvent is at least one of deionized water, PBS buffer and cell culture medium;

The gelatin and the sodium alginate are prepared by a high-speed mixing process at a speed of 1000 to 10,000 revolutions per minute;

The gelatin may be prepared by a gelatin Arabic-gum complex coalescence reaction, and the specific steps of preparing such gelatin may be as follows: 3˜5 grams of A-type gelatin, 0.2˜0.5 grams of Arabic-gum, and 0.5˜1.0 grams of Planic F127 are added sequentially to 200 ml of a mixture of water and alcohol (the volume ratio is in the range of 1:2˜2:1), the solution is dissolved with stirring at 50° C.˜60° C., the pH of the solution is adjusted to 6.2˜6.7 by titration with 1 M hydrochloric acid, and then is cooled down to room temperature, 10 μm˜50 μm gelatin microsphere is obtained.

In above method step S2, the construction of tissue/organ comprises at least one construction of a heart, a liver, a kidney, a pancreas and a brain;

The size of the construction of tissue/organ is 500 μm to 100 mm.

In above method step S3, printing the substructure according to the following step of 1) and/or 2) below:
1) Printing a specific physiological or pathological construction using another cell-laden bioink;
2) Preparing and Printing a sacrificial ink in which an angiogenic cell is laden to construct a sophisticated vascularized channel with a diameter of 100 μm to 5 mm.

The number of the printing stages is determined based on the specific construction of the target tissue/organ model, e.g., when constructing a cardiac chamber with vascularized channel, secondary stage printing is processed, i.e., secondary substructure is printed based on the step described above in 2), and when constructing a brain glioma model, tertiary stage printing is processed, i.e., secondary and tertiary substructures are printed sequentially based on the steps described above in 1) and 2).

In above method step S4, a method for cross-linking the whole construction is at least one of photo cross-linking, thermal (temperature) cross-linking, ionic cross-linking, and enzyme cross-linking and covalent cross-linking;

A method for removing the suspension medium is at least one of temperature change, shaking, washing, enzymatic dissolution, and other ways.

In the method above, when printing the substructure in the step of 2), step S4 further comprises a step of removing the sacrificial ink;

The step of removing the sacrificial ink is at least one of a temperature change, a pH change and an ionic action.

When the suspension medium or the sacrificial ink is a temperature-sensitive gel material, e.g., comprising gelatin, Pluronic-F127 gelatin, it may be dissolved at the gel temperature by utilizing the temperature-sensitive nature of its “gel-sol” transition.

### BRIEF DESCRIPTION OF THE DRAWINGS

FIG. 1 shows a schematic diagram of a cell-laden gel microsphere bioink employed in the present invention, in which, 1 denotes the cell-laden gel microsphere, 2 denotes a cell within the gel microspheres, and 3 denotes the continuous phase gel material surrounding the gel microspheres.

FIGS. 2a-2d shows a characterization of the cardiomyocyte-laden gel microsphere bioink prepared in Example 1 of the present invention, wherein FIG. 2a shows a picture of the gel microsphere obtained by using a T-type microfluidic device, FIG. 2b shows the results of live/dead cell staining of the cardiomyocyte-laden gel microsphere (live cell in green color, and dead cell in red color), FIG. 2c shows the sophisticated lattice structure printed by using the gel microsphere bioink, and FIG. 2d is a partially enlarged view in FIG. 2c.

FIGS. 3a and 3b shows the characterization of the rheological property of cardiomyocyte-laden gel microsphere bioink prepared in Example 1 of the present invention, wherein FIG. 3a shows the variation curve of viscosity with shear rate, and FIG. 3b shows the variation curve of energy storage modulus under alternating high and low strains.

FIG. 4 shows a flow chart of in vitro bionic construction of a cardiac chamber with vascularized channel in embodiment 1 of the present invention, wherein 4 denotes a cardiac chamber structure and 5 denotes a vascular channel.

FIG. 5 shows a flowchart of construction of a bionic brain glioma model in vitro in embodiment 2 of the present invention, wherein 6 denotes nerve tissue, 7 denotes brain glioma structure, and 8 denotes a vascularized channel.

### DETAILED DESCRIPTION

The experimental methods used in the following embodiments are conventional if not otherwise specified.

The materials, reagents and the like used in the following embodiments are commercially available if not otherwise specified.

### EXAMPLE 1: IN VITRO BIONIC CONSTRUCTION OF A CARDIAC CHAMBER WITH VASCULARIZED CHANNEL

### 1. Preparation of Bioink (Cardiomyocyte-Laden)

Cardiomyocytes and vascular endothelial cells derived from pluripotent stem cells were obtained by culturing in vitro and inducing differentiation of human induced pluripotent stem cells, in which gelatin methacryloylated (GelMA) capable of photo-cross-linking was used as the gel microsphere material.

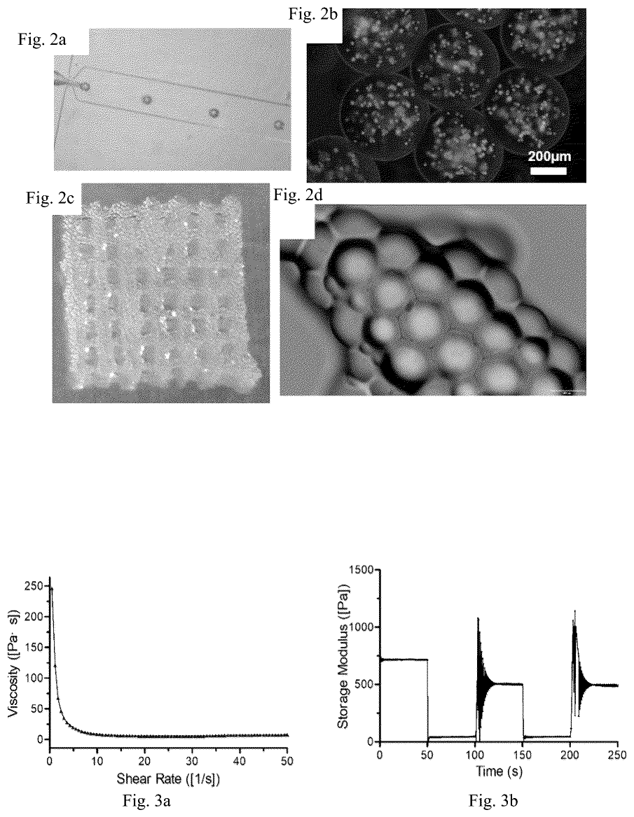

Using a T-type microfluidic device, a 7.5 wt % GelMA solution containing cardiomyocytes with a density of 1×10⁷ cells/mL was passed into the dispersed phase inlet of the T-type microfluidic device at a flow rate of 0.5 mL/h, and mineral oil containing 10% span 80 (span 80, surfactant) was passed into the continuous phase inlet of the T-type microfluidic device at a flow rate of 6.0 mL/h, and photocross-linking was performed in the outlet of the chip to obtain gel microsphere with diameter of 400 μm˜450 μm (FIG. 2a). By live/dead cell staining, the survival rate of cardiomyocytes within the GelMA gel microspheres prepared according to this embodiment reaches 90% or more (FIG. 2b).

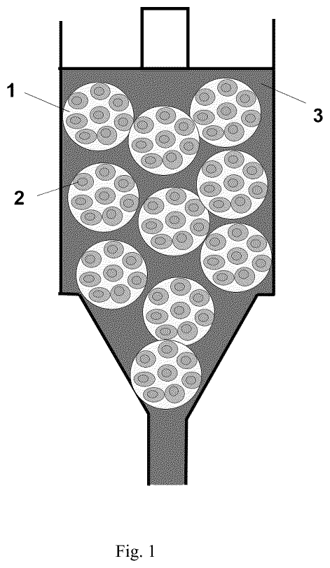

The cardiomyocyte-laden gel microsphere was treated at 4° C. sequentially by washing, filtration, centrifugation, etc., to remove mineral oil in the gel microsphere, and a type I rat tail collagen and a Matrigel solution were mixed into it at a volume ratio of 1:1, to obtain the gel microsphere ink, the structure of which is shown in FIG. 1, wherein the volume content of the gel microsphere in the gel microsphere ink was 50%, cell density within the gel microsphere was 1×10⁷ cells/mL, and mass-volume concentration of the collagen and the Matrigel gel material, as the continuous phase, was 5 mg/mL.

It is shown in experiment that these GelMA gel microsphere inks have good printing performance and can be printed into sophisticated lattice architecture (FIG. 2c) with uniform and stable filament out (FIG. 2d).

Through rheological testing, it can be found that the GelMA gel microsphere ink prepared in this embodiment exhibits shear thinning (FIG. 3a) in addition to self-healing property (FIG. 3b). Among other things, the self-healing property is not present in conventional GelMA gel bioinks. It should be noted that the lowest concentration of GelMA printing that can be achieved by the existing studies is 75 mg/mL, and the 3D printing bioink based on gel microsphere provided by the present invention is capable of achieving a concentration of 50 mg/mL and lower, which can satisfy the requirements of specific cell for an ultra-soft matrix environment.

### 2. In Vitro Construction of a Cardiac Chamber With Vascularized Channel

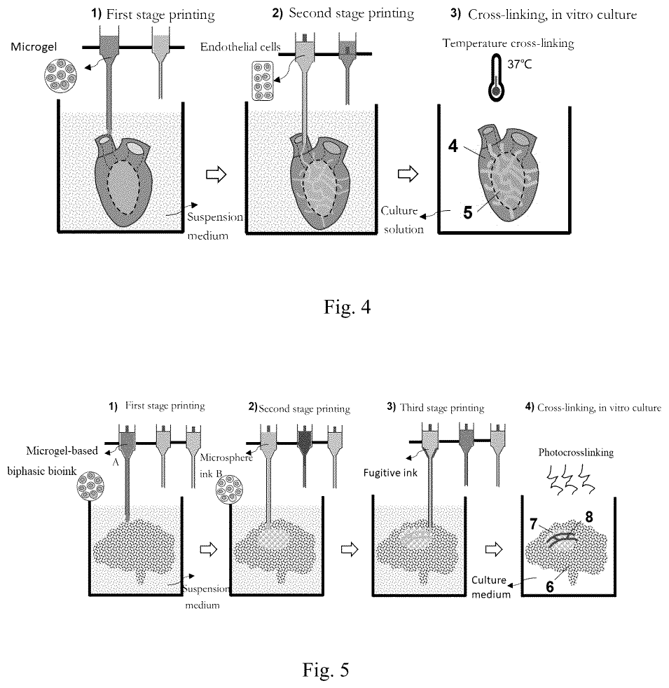

The preparation flowchart is shown in FIG. 4.

The topology of the ventricles and blood vessels was obtained by 3D reconstruction of the human cardiac image, and isotropic reduction was performed to make the outer diameter size of the ventricles to be about 10 mm. Temperature-sensitive gelatin particles were prepared as a suspension medium by a complex coalescence reaction, with particle sizes ranging from 20 μm to 25 μm. The cardiac chamber structure was printed using cardiomyocyte-laden gel microspheres prepared in step 1 in the gelatin suspension medium, with a printing temperature at 22° C., a printing speed of 2 mm/s, and an extrusion speed of 0.5 mm³/s; subsequently, the printed chamber structure was used as a new suspension medium. A gelatin solution with a concentration of 5 wt % was used as a sacrificial ink, in which a vascular network structure of the cardiac chamber was printed at a printing temperature of 20° C., a printing speed of 1 mm/s, and an extrusion speed of 0.2 mm³/s; after the printing was completed, it was placed into the incubator (37° C. and 5% CO₂) and incubated for 30 min, such process has allowed the whole printed structure to undergo a whole temperature cross-linking, and at the same time, a gelatin suspension medium and a sacrificial ink has been dissolved, thereby a cardiac chamber containing hollow channel (outer diameter of the chambers is 10 mm, wall thickness is 1.5 mm) has been constructed; finally, endothelial cell was seeded within the channel by perfusion seeding to form a vascularized channel. Further, the cardiomyocytes were provided with necessary oxygen and nutrients by continuously culture fluid perfusion seeding in vitro to the vascularized channel of the cardiac chamber, and after 1 week of culture, a whole beating of the cardiac chamber appears, such that a vascularized cardiac chamber with mature function and a large-scale size of 10 mm was obtained.

### EXAMPLE 2: IN VITRO BIONIC CONSTRUCTION OF GLIOMA MODEL

### 1. Preparation of Bioink (Neural Cell-Laden)

Patient-derived glioma cells were used for culture and expansion in vitro, while human-derived induced pluripotent stem cells have induced and differentiated into neuronal cells and endothelial cells using a co-axial flow focusing type microfluidic device. The neural cells suspension, hyaluronic acid solution with a mass fraction of 10.0% and Matrigel solution (purchased from BD) with a volume fraction of 40% were homogeneously mixed in a ratio of 1:2:1, such that final density of neural cells was 2×10⁶ cells/mL. The neural cell-laden hyaluronic acid/Matrigel solution was passed through a dispersed phase inlet of the co-axial flow focusing type microfluidic device at a flow rate of 1 mL/h; a mineral oil containing 2% span 80 was passed into a continuous phase inlet of the co-axial flow focusing type microfluidic device at a flow rate of 10.0 mL/h, such that a gel microsphere with diameter of 100 μm˜150 μm was obtained. By live/dead staining, a survival rate of neuronal cell within the hyaluronic acid/Matrigel gel microsphere prepared in this embodiment reached 80% or more.

The neural cell-laden hyaluronic acid/Matrigel gel microsphere was sequentially treated by washing, filtration, centrifugation, etc., to remove the mineral oil in the gel microsphere, and a gelatin methacrylate gelatin (GelMA) solution was mixed to it in a volume ratio of 5:4, to obtain a neural cell-laden gel microsphere ink, and the structural schematic is shown in FIG. 1, in which the volume content of the gel microsphere in the gel microsphere ink is 55%, the cell density within the gel microsphere is 2×10⁶ cells/mL, and the mass-volume concentration of the GelMA gel material as the continuous phase is 25 mg/mL.

Through rheological testing, the hyaluronic acid/Matrigel gel microsphere ink prepared in this embodiment exhibits self-healing property in addition to shear thinning.

### 2. Preparation of Bioink (Glioma Cell-Laden)

A glioma cell suspension, a hyaluronic acid solution with a mass fraction of 10.0% and a fibrinogen solution with a mass fraction of 5.0% were homogeneously mixed in a ratio of 1:3:1, such that a density of glioma cells have rearched 5×10⁶ cells/mL. A glioma cell-laden hyaluronic acid/fibrinogen solution was passed into a dispersed phase inlet of the co-axial flow focusing type microfluidic device at a flow rate of 0.2 mL/h and a mineral oil containing 5% span 80 was passed into a continuous phase inlet of the co-axial flow focusing type microfluidic device at a flow rate of 4.0 mL/h, such that a gel microsphere with diameter of 450 μm˜500 μm was obtained. By live/dead staining, the survival rate of neuronal cell within the hyaluronic acid/fibrinogen gel microsphere prepared in this embodiment reached 95% or more.

The glioma cell-laden hyaluronic acid/fibrinogen gel microsphere was sequentially washed, filtered and centrifuged etc. to remove mineral oil in the gel microspheres, and gelatin methacrylate gelatin (GelMA) solution was mixed to it in a volume ratio of 3:2 to obtain a glioma cell-laden gel microsphere ink, and structural schematic is shown in FIG. 1, in which a volume content of gel microsphere in the gel microsphere ink is 60%, the cell density within the gel microsphere is 5×10⁶ cells/mL, and the mass-volume concentration of the GelMA gel material as the continuous phase is 10 mg/mL.

Through rheological testing, the hyaluronic acid/fibrinogen gel microsphere ink prepared in this embodiment exhibits self-healing property in addition to shear thinning.

### 3. In Vitro Bionic Construction of Brain Glioma Model

The preparation flow chart is shown in FIG. 5.

The brain structure containing vascularized channel and glioma was obtained by three-dimensional reconstruction of the brain imaging data of glioma patient, and isotropic shrinkage was performed and the size of the outer diameter of the brain was scaled to be about 25 mm. Sodium alginate particle was prepared as a suspension medium by high-speed stirring at low temperature (0˜4° C.), and the size of sodium alginate particles ranges from 10 μm to 50 μm. Brain-like structure was printed by using the neuronal cell-laden gel microsphere ink in sodium alginate suspension medium; subsequently, the printed brain-like structure was used as a new suspension medium for the printing of a secondary stage structure, i.e., the glioma cell was printed by using the glioma cell-laden hyaluronic acid/fibrinogen gel microsphere ink; further, the printed glioma structure was used as a new suspension medium for a tertiary stage structure printing, i.e., gelatin solution (with a concentration of 7.5 wt %) lading endothelial cells (with a cell density of 7.5×10⁶ cells/mL) was used as a sacrificial ink to print vascular network structure. After printing, photo cross-linking was applied for the printed structure as the whole cross-linking. Then, it was incubated in an incubator for 30 min, the gelatin sacrificial ink was dissolved and the suspension medium of sodium alginate was removed so as to a bionic glioma model containing vascularized channel was constructed with a size of 15 mm.

### INDUSTRIAL APPLICATION

The multistage suspension 3D printing method provided by the present invention is based on a gel microsphere ink with both shear thinning and self-healing property, which can be printed and shaped in a suspension medium, which can subsequently be used as a suspension medium for the printing of the next stage of structure, which is suitable for constructing a tissue and organ model with vascularized channel and a heterogeneous cellular structure, and can be used for repair of diseased tissue and organ, discovery and screening of drug, and pathology research modeling, etc. The method provides a new technical means for the construction of tissue and organ with sophisticated function, and also lays the foundation for the future whole-organ printing, which is conducive to promoting the clinical application of engineered tissue/organ in regenerative repair therapy.

1-13. (canceled)

14. A multistage suspension printing method for constructing complex heterogeneous tissue/organ, comprising the steps of:
S1, preparing a bioink, the bioink is formed from a cell-laden gel microsphere which is cross-linked, or is obtained by mixing a cell-laden gel microsphere which is cross-linked with at least one gel material which is not cross-linked;
when the cell-laden gel microsphere which is cross-linked with the gel material which is not cross-linked are included, the cell-laden gel microsphere acts as a dispersed-phase and the gel material acts as a continuous-phase;
S2, printing the bioink in a suspension medium to construct a construction of specific tissue/organ;
S3, processing a second stage or multistage printing within the construction of the tissue/organ obtained in step S2 to form substructures;
S4, the suspension medium is dissolving after cross-linking the whole construction, so as to obtain a tissue/organ model with a sophisticated vascular channel and/or a heterogeneous cellular architecture.

15. The multistage suspension printing method according to claim 14, wherein, in step S1, the cell-laden gel microsphere is prepared according to the following method:
at least one of suspension droplet method culture, ultra-low adherence culture plate, magnetic suspension culture, dynamic rotary culture and micro-fluidic technology;
the cell is at least one type selected from the group consisting of tissue parenchymal cell, pluripotent stem cell, induced pluripotent stem cell, angiogenic cell, stromal cell and tumor cell.

16. The multistage suspension printing method according to claim 14, wherein, the cell density in the cell-laden gel microsphere is 10⁶ cells/mL to 10⁸ cells/mL;
the mass-volume concentration of the gel material in the cell-laden gel microsphere is 10˜100 mg/mL.

17. The multistage suspension printing method according to claim 14, wherein, the mass-volume concentration of the gel material as the continuous-phase is 1 to 100 mg/mL;
the gel material which acts as the continuous-phase may lade cell;
the cell density in the gel material is 10⁶ cells/mL to 5×10⁷ cells/mL.

18. The multistage suspension printing method according to claim 14, wherein, in step S1, both the gel material which is used for the cell-laden gel microsphere and the gel material which is used as the continuous-phase are natural polymer hydrogel and/or synthetic polymer hydrogel;
the natural polymer hydrogel is at least one selected from the group consisting of sodium alginate, gelatin, collagen, Matrigel, chitosan, filipin, hyaluronic acid, fibrinogen, chondroitin sulfate, albumin, and their methacryloylated products;
the synthetic polymer hydrogel is at least one selected from the group consisting of polyethylene glycol, polypropylene alcohol, polyethylene glycol diacrylate, polyethylene oxide, polyacrylamide, polyacrylic acid, polyphosphonitrile, poly-N-isopropylacrylamide-type hydrogels, and their methacryloylated products.

19. The multistage suspension printing method according to claim 14, wherein,
the cell-laden gel microsphere has a diameter of 50 μm to 1000 μm and a volume content of 40% to 100% in the bioink.

20. The multistage suspension printing method according to claim 14, wherein, in step S2, the suspension medium is a hydrogel material with self-healing property, specifically is a supramolecular self-healing hydrogel and/or a gel microsphere construction;
the supramolecular self-healing hydrogel is at least one selected from the group consisting of a cyclodextrin-based supramolecular hydrogel, a DNA supramolecular hydrogel, a polyurethane urea supramolecular hydrogel, a hyaluronic acid-dextran supramolecular hydrogel, a tanshinone II-A polypeptide supramolecular hydrogel, and a graphene composite supramolecular hydrogel;
the microgel construction has a size of 1 μm to 50 μm;
the microgel construction is at least one selected from the group consisting of Carbomer, gelatin and sodium alginate.

21. The multistage suspension printing method according to claim 20, wherein, the Carbomer is an acrylic cross-linking resin obtained by cross-linking pentaerythritol with acrylic acid, and solvent is at least one selected from the group consisting of deionized water, PBS buffer and cell culture medium;
the gelatin and the sodium alginate are prepared by a high-speed mixing process at a speed of 1000 to 10,000 revolutions per minute;
the gelatin is prepared by a gelatin Arabic-gum complex coalescence reaction.

22. The multistage suspension printing method according to claim 14, wherein, in step S2, the construction of tissue/organ comprises at least one construction of a heart, a liver, a kidney, a pancreas and a brain;
the size of the construction of tissue/organ is 500 μm to 100 mm.

23. The multistage suspension printing method according to claim 14, wherein, in step S3, the printing method of printing the substructure further comprising the following step of 1) and/or 2) below:
1. printing a specific physiological or pathological construction using another cell-laden bioink;
2. preparing and printing a sacrificial ink in which an angiogenic cell is laden to construct a sophisticated vascular channel with a diameter of 100 μm to 5 mm.

24. The multistage suspension printing method according to claim 14, wherein, in step S4, a method for cross-linking the whole construction is at least one selected from the group consisting of photo cross-linking, temperature cross-linking, ionic cross-linking, enzyme cross-linking, and covalent cross-linking methods;
a method for removing the suspension medium is at least one selected from the group consisting of temperature change, shaking, washing, and enzymatic dissolution.

25. The multistage suspension printing method according to claim 23, wherein, when printing the substructure in the step of 2), step S4 further comprises a step of removing the sacrificial ink;
the step of removing the sacrificial ink is at least one selected from the group consisting of a temperature change, a pH change and an ionic action.

26. A tissue/organ model with the sophisticated vascular channel and the heterogeneous cellular architecture constructed by the method of claim 14.
